(19) United States
(12) Patent Application Publication
(10) Pub. No.: US 2007/0098782 A1
(43) Pub. Date: May 3, 2007

(54) Ramipril Formulation

(75) Inventors: Paul Harrison, Dublin (IE); Anna Marie Power, Dublin (IE)

Correspondence Address: STERNE, KESSLER, GOLDSTEIN & FOX P.L.L.C., 1100 NEW YORK AVENUE, N.W., WASHINGTON, DC, 20005, US

(73) Assignee: Selamine Limited, Dublin (IE)

(21) Appl. No.: 11/508,916

(22) Filed: Aug. 24, 2006

Related U.S. Application Data
(63) Continuation-in-part of application No. 11/273,575, filed on Nov. 15, 2005.

(30) Foreign Application Priority Data
Oct. 28, 2005 (GB) GB 0522047.0

(51) Int. Cl. A61K 31/403; A61K 9/48; A61K 9/20

(52) U.S. Cl. 424/451; 424/464; 514/412

(57) ABSTRACT

A Ramipril formulation which is suitably stabilised to control the degradation to the active metabolite ramiprilat.

### FIELD OF INVENTION

The present invention relates to a dosage form of Ramipril and also to methods of use. In particular, although not exclusively, the present invention relates to stability of formulations for treating or preventing various disease states involving the administration of Ramipril.

### BACKGROUND OF THE INVENTION

Ramipril, the United States Adopted Name (USAN) for (2S,3aS,6aS)-1[(S)-N-[(S)-1-carboxy-3-phenylpropyl] alanyl] octahydrocyclopenta[b]pyrrole-2-carboxylic acid, 1-ethyl ester (CAS Number 087333-19-5) is an angiotensin converting enzyme (ACE) inhibitor having the chemical structure shown below (I).

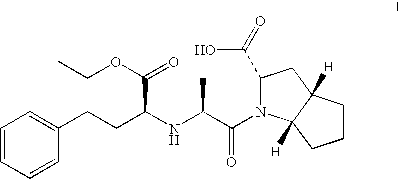

Ramipril and its acid are taught in EP 0 097 022. Ramipril has been used for the treatment of hypertension, heart failure, stroke, myocardial infarction, diabetes and cardiovascular disease. Ramipril may also reduce the risk of further strokes, heart attacks and cognitive impairment among stroke patients. It is commercially available at 1.25 mg, 2.5 mg, 5 mg and 10 mg strengths.

Ramipril is defined in official monographs in both the United States Pharmacopeia and the European Pharmacopoeia. In the European Pharmacopoeia 14 impurities are categorised and labelled as impurities A-N. Impurities A, B, C and D are defined as qualified impurities with impurities E to N being classed as ‘other detectable impurities’. Different limits have been applied to the two sets of impurities. To fulfil the United States standard, only impurities A, B, C and D require quantification. Of the 14 impurities that are named in the European Pharmacopoeia only two are identified as potential degradation products: impurities D and E.

Impurity D, ramipril diketopiperazine, is not active as an ACE inhibitor whereas impurity E, ramipril diacid or ramiprilat, is up to 6 times more potent as an ACE inhibitor than the parent compound ramipril. Ramipril is converted in vivo to ramiprilat and can therefore be considered to be a prodrug of ramiprilat.

Ramiprilat is formed in vivo by ester hydrolysis to this active diacid from ramipril. By the very nature of the compound it is therefore inherently designed to be sensitive to hydrolysis. It is important, when considering the formulation of such a compound that the potential hydrolysis is minimised by design, so that an adequate potency of the active ingredient in the formulation is maintained over the shelf life of the product.

This has traditionally been achieved by excluding water from the formulation and thus preventing hydrolysis of the ramipril to its degradation products. The first choice to a formulator to prevent hydrolysis is, therefore, to develop a dry product for oral administration usually in a tablet or capsule. Indeed such a finding is disclosed in WO2004/064809, where it is claimed that formulations need to be below 5.5% moisture content in order to be stable. Hence, it is desirable to avoid unnecessary or excessive contact of ramipril with water during the process of manufacture of a solid dosage form.

Integral mixing of the components of a solid dosage form can be carried out on dry components, and hence direct compression has become a standard for tablet formulation. Wet granulation methods and spray granulation methods are also known and offer additional options for mixing of tablet components. However, such methods are to be avoided if there is risk of damage to or degradation of components due to contact with solvents used in the granulation.

A commercially viable shelf life of a formulated product would be considered to be 2 years or greater, and an acceptable potency over this shelf life would be 95 to 105%. This potency limit is applied in most European Pharmacopoeias, except where a compound is subject to substantial degradation such as Amoxycillin where a 90% potency lower limit applies.

In a recent communication from the British Pharmacopoeia, it was noted that the considered acceptable potency range of ramipril in a formulated product over its shelf life, was set between 90-105%. Standard potency limits have not, therefore, been applied, with the implication that ramipril is less stable in tablet or capsule formulations than the majority of products. It would, therefore, be desirable to develop a stable formulation that can comply with the 95-105% potency range over the expected shelf life of the product.

As a result of the use of dry formulation techniques and the prevention of hydrolysis, the major degradation product identified in the British Pharmacopoeia is the diketopiperazine derivative (impurity D). The limits imposed by the British Pharmacopoeia on the diketopiperazine derivative infer that the loss in potency over the shelf life of the product would be expected to be due to the conversion of ramipril to the diketopiperazine degradation product. A limit of 8% and 6% for this degradant is applied to the capsule and tablet formulation respectively, and therefore by simple mass balance, the potency could fall below the standard lower limit of 95%. The limit imposed on other impurities including ramiprilat (impurity E) is set at levels below 0.5% and, therefore, such impurities as degradation products are considered to be undesirable.

Various Ramipril formulations are known in the art. Such formulations can be found in, for example, U.S. Pat. No. 4,743,450, U.S. Pat. No. 6,555,551, U.S. Pat. No. 2005/0169981, WO 2004/064809, U.S. Pat. No. 2005/0069586, U.S. Pat. No. 2003/0215526, WO 05/041940, and WO 03/059388. The present application does not concern these known formulations.

Degradation of pharmaceutically active compounds is of concern to both medical practitioners and to the community at large. If significant degradation takes place between manufacture and administration of an active then suboptimal dosing is highly likely. For actives used in the treatment of hypertension and cardiovascular disease dosing accuracy is of tantamount importance as ineffective treatment is likely to result in life-threatening complications.

It would be useful if there were a formulation of Ramipril that avoids significant degradation to inactive impurities.

It is an object of the invention to overcome the disadvantages associated with present ramipril formulations or to at least provide the public with a useful alternative.

### SUMMARY OF THE INVENTION

Accordingly, in a first aspect, the present invention provides a solid dosage form comprising ramipril and a pharmaceutically acceptable carrier, wherein the ramipril is in the form of a ramipril salt.

Preferably, at least 50% of the ramipril is in the form of a ramipril salt.

There is also provided a solid dosage form comprising ramipril and a pharmaceutically acceptable carrier, wherein at least 50% of the ramipril is in the form of a sodium or potassium ramipril salt and the pharmaceutical carrier is selected from the group consisting of calcium sulphate, calcium carbonate and a mixture thereof.

As described in more detail below, it has been found that by providing ramipril in the form of a ramipril salt, degradation to the inactive impurities can be greatly decreased.

More preferably at least 70%, more preferably at least 80%, more preferably at least 85%, further preferably at least 90%, more preferably at least 95%, further preferably at least 98% of the ramipril is in the form of a ramipril salt.

In preferred embodiments, the ramipril salt is selected from a salt of an alkali metal and a salt of an alkali earth metal. Preferably, the salt is selected from the lithium, calcium and potassium salts. Preferably, the salt is the sodium salt.

Preferably, the solid dosage form is in the form of a tablet. Alternatively, the solid dosage form is in the form of a capsule.

In another aspect, the present invention provides a method of making a ramipril formulation, comprising obtaining a ramipril salt and incorporating the ramipril salt into the formulation.

In preferred embodiments at least 50% by weight of the ramipril is in the form of a ramipril salt.

There is also provided a method of making a ramipril formulation, comprising obtaining a ramipril salt and incorporating the ramipril salt into the formulation, wherein at least 50% by weight of the ramipril is in the form of a sodium or potassium ramipril salt and wherein the formulation comprises a pharmaceutical carrier selected from the group consisting of calcium sulphate, calcium carbonate and a mixture thereof.

Preferably, the formulation is in solid dosage form, further preferably a tablet. Alternatively, the solid dosage form is a capsule.

Preferably at least 70%, more preferably at least 80%, more preferably at least 85%, further preferably at least 90%, more preferably at least 95%, further preferably at least 98% by weight of the ramipril is in the form of a ramipril salt.

Preferably, the method comprises:
adding ramipril to an aqueous solvent;
converting the ramipril into a salt of ramipril;
dissolving the salt of ramipril in the aqueous solvent; and
removing the solvent, to yield dried ramipril salt.

In a particularly preferred embodiment, the aqueous solvent consists essentially of water. Alternatively, the solvent comprises a mixture of water and alcohol, more preferably a mixture of water and ethanol.

Preferably, the method comprises dispersing ramipril particles in the aqueous solvent.

In preferred embodiments, the method comprises adding an alkali to the solvent to convert the ramipril into the ramipril salt. Preferably, the method comprises adding sodium hydrogen carbonate to the solvent to convert the ramipril into the ramipril salt.

It is preferred that the method comprises converting at least 50% of the ramipril into the ramipril salt, more preferably at least 70%, more preferably at least 80%, more preferably at least 85%, further preferably at least 90%, more preferably at least 95%, further preferably at least 98%.

In preferred embodiments, the converting comprises maintaining the ramipril in the aqueous solvent in the presence of a metal compound for sufficient time that substantially all the ramipril is converted into ramipril salt.

In a further aspect of the invention there is provided a solid dosage formulation comprising ramipril obtained by making the formulation out of a ramipril preparation, wherein at least 50% of the ramipril in the ramipril preparation is in the form of a ramipril salt.

There is also provided a solid dosage formulation comprising ramipril and a pharmaceutically acceptable carrier, obtained by making the formulation out of a ramipril preparation, wherein at least 50% of the ramipril in the ramipril preparation is in the form of a sodium or potassium ramipril salt and the pharmaceutical carrier is selected from the group consisting of calcium sulphate, calcium carbonate and a mixture thereof.

There is further provided a solid dosage form comprising a ramipril salt, obtained by the methods described herein.

In another aspect there is provided a solid dosage form comprising a sodium or potassium ramipril salt and a pharmaceutical carrier selected from the group consisting of calcium sulphate, calcium carbonate and a mixture thereof, obtained by the methods described herein.

In another aspect the present invention preferably provides a Ramipril formulation which is basic.

All types of dosage forms that can be used for the oral administration of ramipril are anticipated. Examples of such dosage forms include suspensions, solutions, tablets (chewable, dispersible and conventional), capsule formulations, multiparticulate formulations and formulations adapted to control the release of the drug from the oral dosage form, a so called sustained release formulation.

Solid formulations according to the invention preferably give a pH of greater than 7 when made up as a 1% solution in water. Any formulations having this property are said to be basic. Liquid formulations according to the invention preferably have a pH greater than 7.

Surprisingly it has been found that formulations which are basic undergo degradation in a different manner from those formulations presently known, i.e. acidic or neutral formulations. The preferred degradation pathway of basic formulations results in ramiprilat whereas other formulations result in the formation of inactive products such as ramipril diketopiperazine.

The altered degradation pathway is beneficial in the case of ramipril formulations because the product of the altered degradation pathway is an active metabolite of the drug. Degradation over time to other (inactive) products can thus be minimised.

The invention preferably provides Ramipril formulations that display altered degradation pathway to the active metabolite ramiprilat, rather that the inactive diketopiperazine.

The “altered degradation pathway” may be obtained by the inclusion of stabilisers in the formulation that makes the pH of a 1% solution in water basic in pH, i.e. greater than pH 7.

Preferred formulations according to the invention give a pH of greater than 7.5, more preferably greater than pH 8.

Liquid formulations according to the invention preferably have a pH of greater than 7.5, more preferably greater than pH 8.

The term “stabiliser” means any material that by its inclusion will render the pH of a 1% solution of the formulation basic. The examples of such “stabilisers” include carbonate salts, amino acids with basic side chains, and amines, although many suitable “stabilisers” will be know to those of skill in the art.

Preferred formulations according to the invention include citrate, carbonate salts, arginine, and ethanolamine, ethanolamine being particularly useful for liquid formations. Other examples of “stabilisers” include sodium lauryl sulphate, talc, magnesium stearate, sodium carbonate, sodium bicarbonate, calcium carbonate and salts.

In a further aspect the present invention also relates to a ramipril formulation that demonstrates substantially no degradation to ramipril diketopiperazine during storage.

In preferred embodiments substantially all degradation taking place during storage is to ramiprilat.

The formulations of the invention may contain any suitable pharmaceutical excipients such as binders, coatings, sweeteners, surfactants, lubricants, glidants, fillers, other active ingredients, colorants and any other excipients or additives known to those in the art.

Formulations of the invention may contain buffers that keep the pH of the formulation within an alkaline range even in the presence of significant amounts of acid.

The formulations of the invention help to ensure that patients treated using said formulations receive the dose of ramipril (or ramiprilat) intended by the prescribing physician.

Formulations according to the invention also offer extended shelf lives. Because the efficacy of treatment does not decrease as the formulations of the invention age (or at least decreases at a vastly reduced rate when compared to known formulations) less wastage of expired medicaments occurs. There is, therefore, a concomitant reduction in unit cost for medicaments according to the invention over previously known formulations.

Preferred formulations according to invention give degradation to ramipril diketopiperazine during storage at 25° C. and 60% RH for 3 months of less than 1%, more preferably less than 0.5%.

Further preferred formulations according to invention give degradation to ramipril diketopiperazine during storage at 40° C. and 75% RH for 3 months of less than 4%, more preferably less than 2%.

In a further aspect the present invention also provides a method for treating or preventing a disease in a mammal selected from the group consisting of hypertension, heart failure, stroke, myocardial infarction, diabetes and cardiovascular disease or for reducing the risk of further strokes, heart attacks and cognitive impairment among stroke patients comprising administering to a mammal in need of such treatment a formulation according to the present invention.

In some embodiments the mammal is a non-human animal.

The present invention also provides the use of a formulation according to the present invention in the manufacture of a medicament for the treatment of hypertension, heart failure, stroke, myocardial infarction, diabetes and cardiovascular disease or for reducing the risk of further strokes, heart attacks and cognitive impairment among stroke patients.

In preferred embodiments the medicament is in the form of a capsule or tablet. However other embodiments include liquid formulations such as suspensions and syrups.

In a further aspect, this invention provides a therapeutic package suitable for commercial sale, comprising a container, a Ramipril formulation according to the invention, and, associated with said container, notice advising of extended shelf life.

For purposes of this invention Ramipril may be administered alone or in combination with other therapeutic agents. In one embodiment Ramipril is co-administered with a diuretic agent, preferably the diuretic is selected from hydrochlorothiazide or piretanide.

Ramipril is typically present in formulations according to the invention in an amount of from about 1.25 mg to about 10 mg. Other formulations may have 2.5 mg or 5 mg per tablet. The amount of active can be adjusted to be outside these limits depending, for example, on the size of the animal subject being treated (e.g., a horse). The term ‘Ramipril’ includes all the pharmaceutically acceptable versions thereof, e.g. salts, esters, clathrates thereof, and also anhydrous as well as hydrated forms.

In another aspect the invention provides a method for the manufacture of a ramipril formulation including the step of adding at least one basic compound. Basic compounds are known to those of skill in the art and suitable examples are included in the examples as well in this specification. The invention includes within its scope the manufacture of ramipril formulations using any suitable basic compound.

Various aspects of the invention will now be described with reference to examples.

### EXAMPLES

The following examples are provided to illustrate the invention only and should not be construed as limiting the scope of the invention as claimed herein. Some of the Example formulations set out herein fall within the scope of the invention as claimed.

The formulations herein may be varied, that is additions and replacement of ingredients with equivalents may be made, without departing from the scope of the invention as herein claimed. For example, the formulation mentioned may advantageously contain citrate salts in place of carbonates and bicarbonates whilst retaining the extended shelf life.

Many of the examples presented focus on the lowest commercial strength, the 1.25 mg, where the highest percentage degradation would be expected (as % w/w with respect to dose). Higher strength products are formulated by adjusting the ratio of the stabiliser to drug substance to minimise the degradation of the drug substance and adjust the pathway so that the active metabolite is produced.

When ramipril (1.25 mg) is simply mixed with the inert substance starch 130 mg and stored in bottles for 1 month at 40° C. 75% Relative humidity, the drug degrades, and approximately 6% of impurity D is recorded. The pH of such a mixture is pH 5.25.

TABLE 1
Stability of starch/ramipril blend in a capsule

| | 1 month | 2 month | 3 month |
|---|---|---|---|
| Assay | 95.7% | 90.5% | 83.9% |
| Diketopiperazine | 5.22% | 10.75% | 14.07% |
| Ramiprilat | 0.35% | 0.37% | 0.42% |

With the inclusion of the base excipients it is possible to reduce the level of the impurity D and if used at increased levels convert the principle degradation product to impurity E ramiprilat as illustrated in the examples.

TABLE 2
Ramipril formulations with the inclusion of base excipients

| Formulation Reference | 1 | 2 | 3 | 4 | 5 | 6 | 7 | 8 |
|---|---|---|---|---|---|---|---|---|
| Ramipril | 1.25 | 1.25 | 1.25 | 1.25 | 1.25 | 1.25 | 1.25 | 1.25 |
| Sodium hydrogen | 0.3 | 0.6 | 0.9 | 1.25 | — | 1.00 | — | 0.83 |
| carbonate | | | | | | | | |
| Sodium carbonate | — | — | — | — | — | — | 0.625 | — |
| Calcium carbonate | — | — | — | — | — | — | — | 72.9 |
| Microcrystalline cellulose | — | — | — | — | — | — | 46.00 | — |
| Calcium phosphate | — | — | — | — | — | 100.0 | — | — |
| dihydrate | | | | | | | | |
| Povidone k29/32 | — | — | — | — | — | — | — | 0.67 |
| Sodium starch glycollate | — | — | — | — | — | — | — | 4.17 |
| Sodium lauryl sulphate | — | — | — | — | — | 0.5 | — | — |
| I-Arginine | — | — | — | — | 0.9 | — | — | — |
| Calcium sulphate | 114.2 | 114.2 | 114.2 | 114.2 | 114.2 | — | — | — |
| Anhydrous lactose | — | — | — | — | — | — | 40.00 | — |
| Starch pregelatinised | 13.00 | 13.00 | 13.00 | 13.00 | 13.00 | — | — | — |
| L-HPC | — | — | — | — | — | 4.0 | — | — |
| Potato starch | — | — | — | — | — | — | 23.00 | — |
| Maize starch | — | — | — | — | — | 15.0 | — | — |
| Iron oxide red | 0.13 | 0.13 | 0.13 | | 0.13 | — | — | — |
| Silicon dioxide | — | — | — | — | — | — | 0.4 | — |
| Ethanol/water 1:1 | (q.s) | (q.s) | (q.s) | (q.s) | (q.s) | — | — | — |
| Water | | | | | | q.s | — | — |
| Talc | — | — | — | — | — | — | — | 2.09 |
| Sodium stearyl fumarate | 1.30 | 1.30 | 1.30 | 1.30 | 1.30 | | — | — |
| Magnesium stearate | — | — | — | — | — | 1.30 | 1.30 | 0.83 |

Condition 40° C. 75% RH 14 days

| | | | | | | | | |
|---|---|---|---|---|---|---|---|---|
| Impurity D | 25% | 6.5% | 0.75% | 0.3*% | 0.48% | 0.49% | 0.36% | 0.11% |
| Impurity E | 2.5% | 2.1% | 1.1% | 2.0*% | 0.50% | 0.51% | 0.15% | 5.6% |
| pH 1% | 6.94 | 7.36 | 7.75 | 8.79 | 7.87 | 8.26 | 8.07 | 9.19 |

*1 month

The impurity levels reported in the examples above are the levels of impurity when stored in bottles for 14 days at 40° C. 75% relative humidity, with the exception of formulation 4 which was stored for 1 month at the same conditions.

All the examples in table 2 were manufactured on a small scale conventionally either by simply screening and blending the ingredients and then compressing, or if water or water ethanol mixture was used, screening, mixing, granulating drying in fluid bed drier, screening blending and compressing. These two processes direct blending and granulating and blending can be considered to be conventional granulation.

Preferably wet granulation is used to formulate basic formulations according to the invention to ensure that the principle degradation product is ramiprilat.

The batches reported in the following examples were manufactured on a small scale at around 300 g in a Cryto Peerless granulator. Where wet granulation was required, the granule was dried in an Aeromatic Strea 1. The dryer was set at 55° C. and drying was continued until outlet temperature reach approximately 42° C.

Samples of the granule produced were filled into 60 ml HDPE bottles with 33 mm necks and a screw caps and placed on stability at 40° C. 75% RH.

The ramipril raw material used was commercially sourced from Neuland.

The related substances were determined at the time points specified using the standard methods of analysis for this drug.

The only exceptions to the small scale examples were
(i) STD formulations reported: These were manufactured at 78 kg using a Diosna granulator and a Vector fluid bed drier,
(ii) Capsule Data: These were manufactured as part of a development campaign at Cobalt Canada.

An experiment was carried out in which 50 mg of ramipril was dispersed in 50 ml water and known concentrations of buffer were added. The solutions were placed in a stoppered bottle and stored for 12 hours at 50° C. The results are shown in table 3.

TABLE 3
1 mg/ml Ramipril Solution in Buffer: Principal Impurity after 12 hours at
50° C.

| | Amount buffer | | |
|---|---|---|---|
| Buffer | added | Impurity E | Impurity D |
| Sodium carbonate | 0.24 mg/ml | 9.9% | 0.22% |
| Sodium carbonate | 0.74 mg/ml | 11.3% | 0.19% |
| Sodium carbonate | 1.00 mg/ml | 15.2% | 0.17% |
| L arginine | 1.44 mg/ml | 13.7% | 0.16% |
| Sodium citrate | 0.72 mg/ml | 8.2% | 0.22% |
| Sodium citrate | 1.00 mg/ml | 8.4% | 0.23% |
| Sodium citrate | 1.90 mg/ml | 7.5% | 0.20% |

It was surmised from the above experiment that ramiprilat (impurity E) would be the principal ingredient when hydrolysis occurred in an alkali environment, and from the stability in a capsule that the diketopiperazine (impurity D) formed in an acid environment.

It was noted that increased levels of the buffer sodium carbonate tended to enhance the levels of ramiprilat impurity, whereas increases in sodium citrate did not substantially alter the level of ramiprilat. It was surmised that the difference in effect of the buffers was pH related. Sodium carbonate is a strong alkali and has little buffering capacity. Increases in the concentration of sodium carbonate will markedly increase the pH of solution, whereas sodium citrate has strong buffering capacity and increases in the concentration of buffer would not significantly increase the pH of solution.

It was therefore inferred that:
The formulation needs to be in an alkaline to ensure that the ramiprilat is formed in an aqueous environment.
Excess of alkali in the formulation will likely to enhance the hydrolysis to ramiprilat.

It can be seen from the solution results in table 3 that the concentration of the alkaline agent is important when strong alkalis such as sodium carbonate are used. Trial batches were prepared initially with the weaker alkali, sodium bicarbonate. The stability of formulations was then compared where sodium bicarbonate was replaced with arginine, sodium carbonate, and the buffer sodium citrate.

TABLE 4
Formulations of Ramipril tablets containing the Stabilising Agent Sodium
Bicarbonate

| Formulation | D | E | F | STD | Formulation | G | K |
|---|---|---|---|---|---|---|---|
| Ramipril | 1.25 | 1.25 | 1.25 | 1.25 | Ramipril | 1.25 | 1.25 |
| Sodium hydrogen | 0.3 | 0.6 | 0.9 | 1.25 | Sodium hydrogen | 1.25 | 1.25 |
| carbonate | | | | | carbonate | | |
| Sodium carbonate | — | — | — | | Microcrystalline | 46.00 | 46.00 |
| | | | | | cellulose | | |
| Calcium sulphate | 114.20 | 114.20 | 114.20 | 113.20 | Starch Potato | 23.00 | 23.00 |
| Starch | 13.00 | 13.00 | 13.00 | 13.00 | Calcium | 40.0 | — |
| pregelatinised | | | | | carbonate | | |
| Iron oxide red | 0.13 | 0.13 | 0.13 | — | Anhydrous | — | 40.00 |
| | | | | | lactose | | |
| Ethanol/water 1:1 | (32) | (32) | (32) | (32) | Silicon dioxide | 0.4 | 0.4 |
| Sodium stearyl | 1.30 | 1.30 | 1.30 | 1.30 | Magnesium | 1.30 | 1.30 |
| fumarate | | | | | stearate | | |
| Total | 130.18 | 130.48 | 130.78 | 130.00 | | 130.18 | 130 |
| PH 1% | 6.9 | 7.4 | 7.8 | | pH 1% | 9.4 | 8.1 |
| PH 5% | 7.0 | 7.5 | 7.7 | | pH 5% | 8.8 | 7.8 |
| Imp D 14 days | 25 | 6.5 | 0.75 | — | Imp D 14 days | 0.48 | 0.64 |
| Imp E 14 days | 2.5 | 2.1 | 1.1 | — | Imp E 14 days | 0.28 | 0.18 |
| Imp D 40 days | 21.7 | 10.0 | 1.6 | 0.20* | Imp D 40 days | 1.4 | 1.84 |
| Imp E 40 days | 2.7 | 3.6 | 2.5 | 1.07* | Imp E 40 days | 1.27 | 1.00 |
| Imp D 150 days | 26.6 | 12.6 | 2.9 | 0.3** | Imp D 150 days | 3.3 | 8.8 |
| ImpE 150 days | 12.9 | 23.5 | 22.1 | 10.1** | ImpE 150 days | 14.0 | 16.1 |
| MOM | WG S | WG S | WG S | WG S | MOM | dc | dc |

*= 30 days not 40 days
**= 180 days not 150 days
WGS = wet granulated with ethanol/water mix
DC = direct compression

The results from table 4 indicate:
The principal degradant changes with increases in bicarbonate levels from diketopiperazine to ramiprilat in the formulation with calcium sulphate as the diluent and wet granulated. Examples DEF & STD
The level of the diketopiperazine formed is reduced with increases in bicarbonate levels in the formulation with calcium sulphate as the diluent and wet granulated.

The change in the principle degradant occurs from diketopiperazine to ramiprilat only after long term stability with products manufactured by direct compression

TABLE 5
The Effect of Process on Formulations of Ramipril tablets containing the
Stabilizing Agent Sodium Bicarbonate

| | STD | STD | | | | | |
|---|---|---|---|---|---|---|---|
| Formulation | mixing | mixing 5 | | | | | |
| Reference | 30 secs | mins | Formulation | G | K | G(1) | K(1) |
| Ramipril | 1.25 | 1.25 | Ramipril | 1.25 | 1.25 | 1.25 | 1.25 |
| Sodium | 1.25 | 1.25 | Sodium | 1.25 | 1.25 | 1.25 | 1.25 |
| hydrogen | | | hydrogen | | | | |
| carbonate | | | carbonate | | | | |
| Sodium | | | Microcrystalline | 46.00 | 46.00 | 46.00 | 46.00 |
| carbonate | | | cellulose | | | | |
| Calcium sulphate | 113.20 | 113.20 | Starch Potato | 23.00 | 23.00 | 23.00 | 23.00 |
| Starch | 13.00 | 13.00 | Calcium | 40.0 | — | 40.0 | — |
| pregelatinised | | | carbonate | | | | |
| Iron oxide red | — | — | Anhydrous | — | 40.00 | — | 40.00 |
| | | | lactose | | | | |
| Ethanol/water 1:1 | — | — | Silicon dioxide | 0.4 | 0.4 | 0.4 | 0.4 |
| Sodium stearyl | 1.30 | 1.30 | Magnesium | 1.30 | 1.30 | 1.30 | 1.30 |
| fumarate | | | stearate | | | | |
| Total | 130.00 | 130.00 | Total | 130.18 | 130 | 130.18 | 130 |
| PH 1% | | | pH 1% | 9.4 | 8.1 | | |
| PH 5% | | | pH 5% | 8.8 | 7.8 | | |
| Imp D 7 days | 10.0 | 0.35 | Imp D 14 days | 0.48 | 0.64 | | |
| Imp E 7 days | 1.80 | 1.0 | Imp E 14 days | 0.28 | 0.18 | | |
| | | | Imp D 40 days | 1.4 | 1.84 | 0.34 | 1.50 |
| | | | Imp E 40 days | 1.27 | 1.00 | 0.80 | 0.42 |
| | | | Imp D 150 days | 3.3 | 8.8 | 2.51 | 8.6 |
| | | | ImpE 150 days | 14.0 | 16.1 | 8.1 | 7.7 |
| MOM | WG | WG | MOM | dc | dc | WG | WG |
| | 30 sec | 5 min | | | | | |

The stability results from table 5 indicate:
By increasing the mixing time of the wet granulation it is possible to reduce the total impurities formed and alter the principal impurity from the diketopiperazine impurity to ramiprilat.
By granulating a portion of the granule from formulation G after 14 days, it is possible to reduce the total impurity formed (40 days and 150 days) and make ramiprilat the principal degradant.
By granulating a portion of granule from formulation K after 14 days, it is possible to reduce the total impurity formed

It could be implied from the work shown that the calcium salt is preferable in stabilising the formulation. To test this, an alternative formulation using dibasic calcium phosphate was manufactured.

TABLE 6
Comparison of Formulations of Ramipril tablets containing Sodium
Bicarbonate with either dibasic calcium phosphate or calcium carbonate
as the diluent

| Formulation Reference | A | | B | C | N |
|---|---|---|---|---|---|
| Ramipril | 1.25 | | 2.5 | 10.00 | 1.25 |
| Microcrystalline cellulose | | | | 181.76 | — |
| Sodium hydrogen carbonate | 1.25 | | 1.66 | 5.00 | 1.0 |
| Calcium phosphate dihydrate | 51.0 | | — | — | 300.0 |
| Calcium carbonate | — | | 145.8 | 73.66 | — |
| Povidone | | | 1.33 | | — |
| Sodium starch glycollate | | | 8.33 | | — |
| Silicon dioxide | | | 0.833 | 1.8 | — |
| Potato Starch | 6.25 | | — | | — |
| Maize starch | | | | 90.88 | 15.0 |
| Sodium croscarmellose | | | | 13.6 | — |
| L-HPC | 1.875 | | — | 2.6 | 4.0 |
| Purified water | (37.5) | | (25) | (q.s) | qs |
| Sodium lauryl sulphate | 0.375 | | — | 10.0 | 0.50 |
| Talc | | | 4.17 | — | 2.5 |
| Magnesium stearate | 0.625 | | 1.66 | 1.3 | 1.25 |
| LOD | 1.2% | | 0.8% | 1.3% | 1.4% |
| Total | 62.5 | mg | 166.28 | 130 mg | 125.5 |

| | | | | |
|---|---|---|---|---|
| Hardness | 3 kp | 3 kp | 8 kp | 4 kp |
| Disintegration water | 1 min | 20 | 20 | 2 min |
| | 30 sec | seconds | seconds | |
| Ph 1% solution | 8.2 | 9.2 | 9.22 | 8.3 |
| pH 5% solution | | 8.6 | 8.05 | 7.9 |
| Imp D 14 days % | 18 | 0.11 | 1.09 | 0.49 |
| Imp E 14 days % | 5.0 | 5.57 | 0.43 | 0.51 |
| Imp D 40 days % | | 0.82 | 1.67 | 20.5 |
| Imp E 40 days % | | 7.00 | 0.92 | 6.77 |
| Imp D 150 days % | 50.0 | 0.36 | 3.64 | 26.7 |
| Imp E 150 days % | 5.42 | 39.6 | 4.08 | 31.4 |

The results from table 6 show that
Formulations with dibasic calcium phosphate are less stable than formulations that use alternative calcium salts, such as calcium sulphate & calcium carbonate
The stability of the product is sensitive to increases in the bicarbonate levels rather than calcium carbonate levels as formulation C is more stable when compared with formulation B. Formulation C has a higher percentage of sodium bicarbonate and a lower percentage levels with respect to ramipril.

It is clear from the results presented that the percentage of sodium bicarbonate with respect to ramipril is important to the stability of the product. It therefore follows that there is a need to establish whether this effect is specific to sodium bicarbonate or can be demonstrated by alternative alkalis.

Equivalent formulations to the examples in tables 4 to 6 were manufactured and compared directly against the bicarbonate products.

TABLE 7A
Comparison of Formulations of Ramipril tablets containing a different
Stabilising Agent
Stabilising agents of choice were Sodium Bicarbonate, Arginine, and Sodium
Carbonate.

| Formulation | G | H | J | K | L | M |
|---|---|---|---|---|---|---|
| Ramipril | 1.25 | 1.25 | 1.25 | 1.25 | 1.25 | 1.25 |
| Sodium hydrogen carbonate | 1.25 | — | — | 1.25 | — | — |
| Sodium carbonate | — | 0.625 | — | | 0.625 | |
| I-Arginine | — | — | 0.625 | — | — | 0.625 |
| Microcrystalline cellulose | 46.00 | 46.00 | 46.00 | 46.00 | 46.00 | 46.00 |
| Starch Potato | 23.00 | 23.00 | 23.00 | 23.00 | 23.00 | 23.00 |
| Calcium carbonate | 40.0 | 40.0 | 40.0 | — | — | — |
| Anhydrous lactose | — | — | — | 40.00 | 40.00 | 40.00 |
| Silicon dioxide | 0.4 | 0.4 | 0.4 | 0.4 | 0.4 | 0.4 |
| Magnesium stearate | 1.30 | 1.30 | 1.30 | 1.30 | 1.30 | 1.3 |
| Total | 130.18 | 130.48 | 130.78 | 130 | 130 mg | |
| pH 1% | 9.4 | 9.8 | 9.60 | 8.1 | 8.1 | 7.70 |
| pH 5% | 8.8 | 9.6 | 9.20 | 7.8 | 7.9 | 7.4 |
| Imp D 14 days | 0.48 | 0.32 | 0.53 | 0.64 | 0.36 | — |
| Imp E 14 days | 0.28 | 0.30 | 0.17 | 0.18 | 0.18 | — |
| Imp D 40 days | 1.4 | 0.74 | 1.6 | 1.84 | 0.96 | 2.3 |
| Imp E 40 days | 1.27 | 1.39 | 0.74 | 1.00 | 0.86 | 0.67 |
| Imp D 150 days | 3.3 | 15.7 | 4.8 | 8.8 | 10.8 | 18.4 |
| Imp E150 days | 14.0 | 20.0 | 9.1 | 16.1 | 20.0 | 8.4 |
| MOM | Dc | dc | dc | Dc | dc | Dc |

wet granulated after 14 days and placed on stability

| | | | | | | |
|---|---|---|---|---|---|---|
| Imp D 40 days | 0.34 | 0.41 | 0.85 | 1.50 | 0.24 | 11.2 |
| Imp E 40 days | 0.80 | 0.86 | 0.38 | 0.42 | 0.28 | 0.68 |
| Imp D 150 days | 2.51 | 1.7 | 3.6 | 8.6 | 1.5 | 28.8 |
| Imp E 150 days | 8.1 | 13.7 | 3.9 | 7.7 | 4.5 | 6.4 |

TABLE 7B
Comparison of Formulations of Ramipril tablets containing a different
Stabilising Agent
Stabilising agents of choice were Sodium Bicarbonate, Arginine, and
Sodium Carbonate.

| Formulation Reference | F | Z | Y | |
|---|---|---|---|---|
| Ramipril | 1.25 | 1.25 | 1.25 | |
| Sodium hydrogen carbonate | 0.9 | — | — | |
| Sodium carbonate | — | 0.45 | | |
| I-Arginine | — | — | 0.9 | |
| Calcium sulphate | 114.20 | 114.20 | 114.20 | |
| Starch pregelatinised | 13.00 | 13.00 | 13.00 | |
| Iron oxide red | 0.13 | 0.13 | 0.13 | |
| Ethanol/water 1:1 | (q.s) | (q.s) | (q.s) | |
| Sodium stearyl fumarate | 1.30 | 1.30 | 1.30 | |
| Total | 130.78 | 130 | 130 | mg |

| | | | |
|---|---|---|---|
| pH 1% | 7.8 | 9.0 | 7.9 |
| pH 5% | 7.7 | 9.3 | 8.11 |
| Imp D 14 days | 0.75 | 0.95 | 0.48 |
| Imp E 14 days | 1.1 | 0.13 | 0.50 |
| Imp D 40 days | 1.6 | 0.32 | 1.55 |
| Imp E 40 days | 2.5 | 2.5 | 1.24 |
| Imp D 150 days | 2.9 | 0.49 | 3.17 |
| Imp E 150 days | 22.1 | 23.6 | 13.2 |

The results from the tables 7A and 7B indicate that:
In all examples with arginine, sodium carbonate and sodium bicarbonate, ramiprilat is the principle degradant, with the exception of formulation M where arginine was low in concentration relative to ramipril and lactose was the diluent and formulation J where the product was wet granulated after 14 days with a high diketopiperazine value at granulation stage and lactose was the diluent.
The trends highlighted for sodium carbonate are replicated for the alternative alkalis. An optimal concentration of alkali to ramipril is thus preferred.

Calcium sulphate and calcium carbonate are also preferred. Wet granulation reduces the total impurity when compared to direct compression

Formulations were manufactured with the buffer sodium citrate, which buffers to a pH around 7.8.

TABLE 8
Formulations of Ramipril tablets containing increasing concentration of
Sodium Citrate.

| Ingredient | 1 | | 2 | | 3 | | 4 | | 5 | |
|---|---|---|---|---|---|---|---|---|---|---|
| Ramipril | 1.25 | mg | 1.25 | mg | 1.25 | mg | 1.25 | mg | 1.25 | mg |
| Microcrystalline Cellulose PH101 | 46 | mg | 46 | mg | 46 | mg | 46 | mg | 46 | mg |
| Potato Starch | 15 | mg | 15 | mg | 15 | mg | 15 | mg | 15 | mg |
| Sodium Citrate | 20 | mg | 40 | mg | 60 | mg | 80 | mg | 100 | mg |
| Lactose Anhydrous | 65 | mg | 65 | mg | 65 | mg | 65 | mg | 65 | mg |
| Silica Dioxide | 0.4 | mg | 0.4 | mg | 0.4 | mg | 0.4 | mg | 0.4 | mg |
| Magnesium Stearate | 1.3 | mg | 1.3 | mg | 1.3 | mg | 1.3 | mg | 1.3 | mg |
| Total | 149 | mg | 169 | mg | 189 | mg | 209 | mg | 229 | mg |

After 14 days at 40 C/75 RH 10% ethanol: 10% water granulation

| | | | | | |
|---|---|---|---|---|---|
| Imp D | 19.5 | 16.6 | 18.5 | 18.5 | 15.0 |
| Imp E | 1.06 | 0.89 | 1.02 | 0.86 | 0.41 |

After 14 days at 40 C/75 RH: 10% water granulation

| | | | | | |
|---|---|---|---|---|---|
| Imp D | — | — | 4.94 | 1.88 | 13.7 |
| Imp E | — | — | 0.99 | 0.55 | 0.90 |
| Imp D | 36.8 | 40.6 | 46.7 | 50 | 46 |
| Imp E | 5.0 | 4.2 | 5.0 | 1.4 | 0.21 |

It can be inferred from the results in table 8 that;
Ramiprilat is not the principle degradant when the alkali is replaced by a buffer.

In summary of the above data it is apparent that;
Ramiprilat is the principle degradant when alkaline substances are added, such as arginine, sodium bicarbonate and sodium carbonate.
The ratio of the alkaline substance used to stabilise ramipril is important with regard to degradation pathway, the total impurity levels detected on stability, and the extent of the suppression of the diketopiperazine impurity level.
An alkaline pH of solution is not sufficient alone to induce the degradation pathway to ramiprilat.
Wet granulation reduces the total impurity levels on stability.
Mixing times in granulation affect the stability pathway for the product, and the total impurities.
The diluents calcium sulphate and calcium carbonate are preferred to dibasic calcium phosphate and lactose.

It is believed that ramipril reacts with the alkaline substances to form a salt in situ. The sodium or arginate component of the salt prevents by steric hindrance the degradation pathway to the diketopiperazine.

This would explain why wet granulation affords better stability as the wet granulation process allows the salt to be formed in the granulating solvent. It would also explain why mixing times in granulation may be important. When short mixing times are selected there is not enough time for the salt to fully form. It is, therefore, preferable that the mixing time is sufficient to enable as much as possible of the ramipril to be converted to the salt, sufficient to convert the precentages of ramipril recited in embodiments into the salt form.

It would appear from the stability data presented that the levels of alkali agents are in excess of the molar concentration required to form a stoichiometric salt of ramipril. Granulation process involves the mixing of a number of ingredients and some of these ingredients will dissolve in water used for granulation. The granulation solvent in the powder mix will therefore be a complex solution. It is probable that an excess alkali is required to ensure that the salt is formed in situ.

It also follows that calcium sulphate and calcium carbonate are the preferred excipients, because the microenvironment of the granule, the surface of the material will be alkaline, whereas the microenvironment for lactose and surprisingly dibasic calcium phosphate is acidic. The acid environment of calcium phosphate was first identified by W Dulin (Drug Dev & Ind. Pharmacy 21(4)393-409 (1995)) and was a factor in the stability of bisoprolol. The acid nature of the dibasic calcium phosphate microenvironment reduced the stability of bisoprolol tartrate. It is therefore likely that not all the ramipril is converted to the salt in the acid microenvironment likely in lactose and dibasic calcium phosphate formulations, and therefore the pathway of degradation to the diketopiperazine is not negated.

Preferably, the product utilises sodium bicarbonate as the stabilising agent and calcium sulphate as the major diluent. Calcium sulphate has an advantage over other excipients in that it can absorb water into its structure through the formation of complex hydrates, reducing the amount of free water available for the hydrolysis reaction. It therefore negates the claim that low moisture content is essential for achieving adequate stability for the product. The preferred formulations are stable with up to 8% moisture being detected.

STD formulation used tables 4 & 5 and are fully described in table 10.

TABLE 10
Preferred Formulations of Ramipril

| Formulation Reference | mg/dose | mg/dose | mg/dose | mg/dose |
|---|---|---|---|---|
| Ramipril | 1.25 | 2.5 | 5.0 | 10.0 |
| Sodium hydrogen carbonate | 1.25 | 2.5 | 5.0 | 10.0 |
| Calcium sulphate | 113.20 | 110.7 | 221.4 | 442.8 |
| Starch pregelatinised | 13.00 | 13.0 | 26.0 | 52.0 |
| Ethanol/water 1:1 | — | — | — | — |
| Sodium stearyl fumarate | 1.30 | 1.3 | 2.6 | 5.2 |
| Total | 130.00 | 130.0 | 260.0 | 520.0 |

These have been manufactured at commercial scale 78 kg and the data summary of the stability data is as follows.

Max Moisture Value recorded: 8.1% at 25C 60% RH & 7.1% at 40C 75% RH

Max Diketopiperazine value: 0.3% at 25C 60% RH at 24 months 0.5% at 40C/75% RH 6 months

Minimum Assay at 25C 60% RH=96% at 24 months

Minimum Assay at 40C/75% RH=92% at 6 months.

It can be concluded that the conditions preferred for producing a tablet of ramipril that is stable over its shelf life and where the principal degradant is the “active” metabolite/compound ramiprilat, is to manufacture the product in such a way that it is possible to form a salt of ramipril in situ, by reacting the acid component of ramipril with a suitable alkaline. Preferably the principal excipients in the mixture such as the diluent should not be acidic.

The invention thus provides stable Ramipril-containing formulations together with methods for the manufacture thereof.

1. A solid dosage form comprising ramipril and a pharmaceutically acceptable carrier, wherein at least 50% of the ramipril is in the form of a sodium or potassium ramipril salt and the pharmaceutical carrier is selected from the group consisting of calcium sulphate, calcium carbonate and a mixture thereof.

2. The solid dosage form of claim 1, wherein at least 70% of the ramipril is in the form of a ramipril salt.

3. The solid dosage form of claim 1, wherein at least 80% of the ramipril is in the form of a ramipril salt.

4. The solid dosage form of claim 1, wherein at least 90% of the ramipril is in the form of a ramipril salt.

5. The solid dosage form of claim 1, wherein at least 95% of the ramipril is in the form of a ramipril salt.

6. The solid dosage form of claim 1, wherein at least 98% of the ramipril is in the form of a ramipril salt.

7. The solid dosage form of claim 1, wherein the salt is the sodium salt.

8. The solid dosage form of claim 1 in the form of a tablet.

9. The solid dosage form of claim 1 in the form of a capsule.

10. A method of making a ramipril formulation, comprising obtaining a ramipril salt and incorporating the ramipril salt into the formulation, wherein at least 50% by weight of the ramipril is in the form of a sodium or potassium ramipril salt and wherein the formulation comprises a pharmaceutical carrier selected from the group consisting of calcium sulphate, calcium carbonate and a mixture thereof.

11. The method of claim 10, wherein the formulation is in solid dosage form.

12. The method of claim 11, wherein the solid dosage form is a tablet.

13. The method of claim 11, wherein the solid dosage form is a capsule.

14. The method of claim 10, wherein at least 70% by weight of the ramipril is in the form of a ramipril salt.

15. The method of claim 10, wherein at least 80% by weight of the ramipril is in the form of a ramipril salt.

16. The method of claim 10, wherein at least 90% by weight of the ramipril is in the form of a ramipril salt.

17. The method of claim 10, wherein at least 95% by weight of the ramipril is in the form of a ramipril salt.

18. The method of claim 10, wherein at least 98% by weight of the ramipril is in the form of a ramipril salt.

19. The method of claim 10, comprising:
adding ramipril to an aqueous solvent;
converting the ramipril into a salt of ramipril;
dissolving the salt of ramipril in the aqueous solvent; and
removing the solvent, to yield dried ramipril salt.

20. The method of claim 19, wherein the aqueous solvent consists essentially of water.

21. The method of claim 20, wherein the solvent comprises a mixture of water and alcohol.

22. The method of claim 20 wherein the solvent comprises a mixture of water and ethanol.

23. The method of claim 19, wherein the method comprises dispersing ramipril particles in the aqueous solvent.

24. The method of claim 19, wherein the method comprises adding an alkali to the solvent to convert the ramipril into the ramipril salt.

25. The method of claim 19, wherein the method comprises adding sodium hydrogen carbonate to the solvent to convert the ramipril into the ramipril salt.

26. The method of claim 19, wherein the method comprises converting at least 50% of the ramipril into the ramipril salt.

27. The method of claim 19, wherein the method comprises converting at least 70% of the ramipril into the ramipril salt.

28. The method of claim 19, wherein the method comprises converting at least 80% of the ramipril into the ramipril salt.

29. The method of claim 19, wherein the method comprises converting at least 90% of the ramipril into the ramipril salt.

30. The method of claim 19, wherein the method comprises converting at least 95% of the ramipril into the ramipril salt.

31. The method of claim 19, wherein the method comprises converting at least 98% of the ramipril into the ramipril salt.

32. The method of claim 19, wherein the converting comprises maintaining the ramipril in the aqueous solvent in the presence of a metal compound for sufficient time that substantially all the ramipril is converted into ramipril salt.

33. A solid dosage formulation comprising ramipril and a pharmaceutically acceptable carrier, obtained by making the formulation out of a ramipril preparation, wherein at least 50% of the ramipril in the ramipril preparation is in the form of a sodium or potassium ramipril salt and the pharmaceutical carrier is selected from the group consisting of calcium sulphate, calcium carbonate and a mixture thereof.

34. A solid dosage form comprising a sodium or potassium ramipril salt and a pharmaceutical carrier selected from the group consisting of calcium sulphate, calcium carbonate and a mixture thereof, obtained by the method of claim 10.
